(19) United States
(12) Patent Application Publication
(10) Pub. No.: US 2004/0117888 A1
(43) Pub. Date: Jun. 24, 2004

(54) Protective mask with a side window

(76) Inventor: Anthony Wang-Lee, Yung-Kang City (TW)

Correspondence Address: WEBB ZIESENHEIM LOGSDON ORKIN & HANSON P.C., 700 Koppers Building, 436 Seventh Avenue, Pittsburgh, PA, 15219-1818, US

(21) Appl. No.: 10/385,324

(22) Filed: Mar. 10, 2003

(30) Foreign Application Priority Data
Dec. 20, 2002 (TW) 91220776

(51) Int. Cl. A42B 1/00; A61F 9/00

(52) U.S. Cl. 2/8; 2/9; 2/424

(57) ABSTRACT

A protective mask includes a mask body having a front part formed with a front window, and left and right side parts, each of which is formed with a side window. A front viewing member is mounted on the front window. Two side viewing members are mounted respectively in the side windows of the left and right side parts and permit vision through the side windows. Two side covers are pivoted to the mask body for covering the side windows.

### BACKGROUND OF THE INVENTION

1. Field of the Invention

The present invention relates to a protective mask, more particularly to a protective mask with a side window so as to permit a wide viewing angle.

2. Description of the Related Art

Referring to FIG. 1, a conventional protective mask 1 is shown to include a mask body 11 having left and right side parts 112 (only one is visible) and a front part 111 disposed frontwardly and transversely of and interconnecting the left and right side parts 112 and defining a front window 113. A viewing member 12 is mounted on the front part 111 so as to cover the front window 113 and so as to permit vision therethrough. A liquid crystal display panel 13 is mounted on the mask body 11, and is disposed frontwardly of the viewing member 12.

One disadvantage of the aforesaid conventional protective mask 1 resides in that the user cannot see through the left and right side parts 112, thereby limiting the viewing range and inconveniencing the user.

### SUMMARY OF THE INVENTION

Therefore, the object of this invention is to provide a protective mask formed with a side window so as to overcome the aforesaid disadvantage of the prior art.

Accordingly, a protective mask of the present invention includes: a mask body including left and right side parts and a front part disposed frontwardly and transversely of and interconnecting the left and right side parts, the front part being formed with a front window, at least one of the left and right side parts being formed with a side window; a front viewing member attached to the front part and spanning the front window to cover the front window; and a side viewing member attached to a respective one of the left and right side parts and spanning the side window to cover the side window.

### BRIEF DESCRIPTION OF THE DRAWINGS

Other features and advantages of this invention will become more apparent in the following detailed description of the preferred embodiment of this invention, with reference to the accompanying drawings, in which:

FIG. 1 is a fragmentary sectional side view of a conventional protective mask;

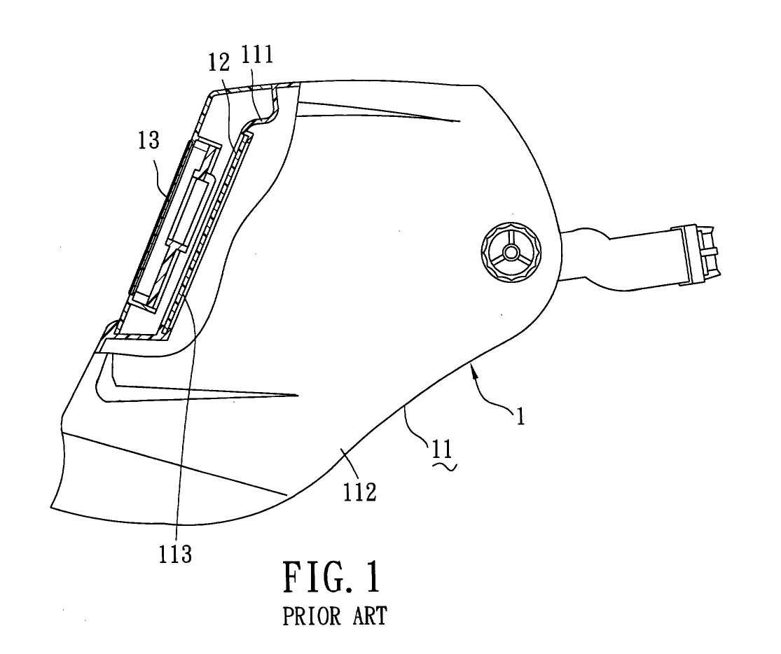

FIG. 2 is an exploded perspective view of the preferred embodiment of a protective mask according to the present invention;

FIG. 3 is a fragmentary sectional side view of the preferred embodiment;

FIG. 4 is a fragmentary sectional side view of the preferred embodiment, illustrating how a side cover is mounted on a mask body to cover a side window;

FIG. 5 is a fragmentary sectional top view of the preferred embodiment, illustrating the side cover in a closed position to cover the side window; and

FIG. 6 is a fragmentary sectional top view of the preferred embodiment, illustrating the side cover in an open position to permit vision through the side window.

### DETAILED DESCRIPTION OF THE PREFERRED EMBODIMENT

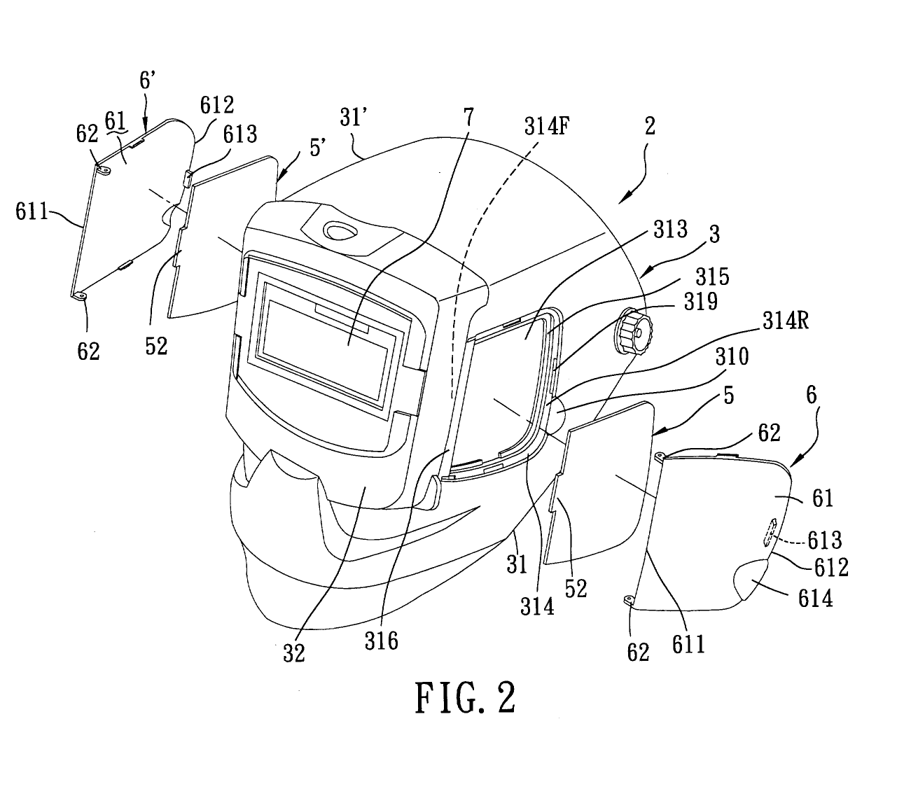

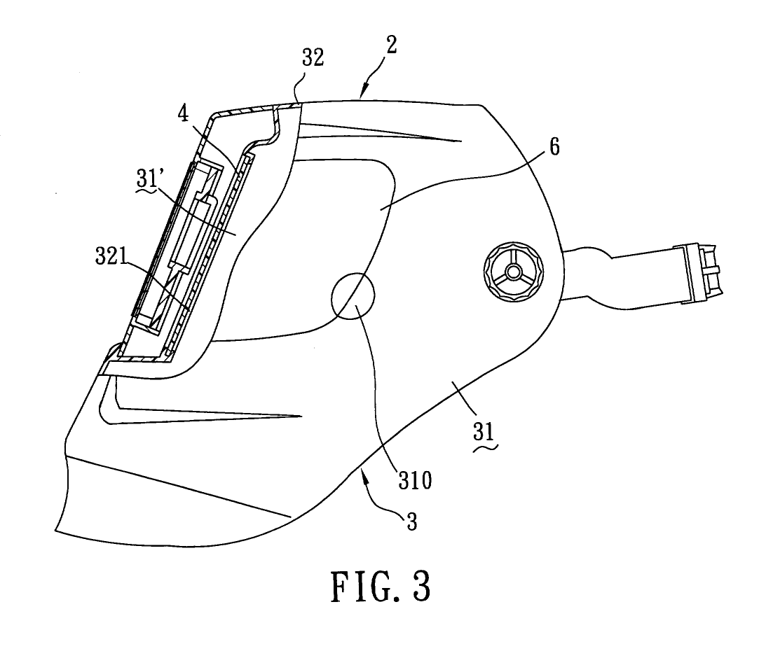

Referring to FIGS. 2, 3 and 4, the preferred embodiment of a protective mask 2 according to the present invention is shown to include a mask body 3, a front viewing member 7, two side viewing members 5, 5′, and a covering unit 6, 6′ including left and right side covers 61.

The mask body 3 is adapted to be mounted detachably on a helmet (not shown), and includes left and right side parts 31,31′ and a front part 32 disposed frontwardly and transversely of and interconnecting the left and right side parts 31,31′. The front part 32 is formed with a front window 321. Each of the left and right side parts 31,31′ is formed with a side window 313.

The front viewing member 7, such as a tinted glass, is attached to the front part 32, and spans the front window 321 to cover the same.

Each of the side viewing members 5, 5′ is attached to a respective one of the left and right side parts 31,31′ and spans the side window 313 to cover the same.

Each of the side windows 313 is defined by a window-confining wall 314. Each of the left and right side parts 31,31′ of the mask body 3 is further formed with an abutting flange 315 projecting from the window-confining wall 314 in a transverse direction relative to the window-confining wall 314 into the side window 313 in such a manner that the side viewing member 5 abuts against the abutting flange 315.

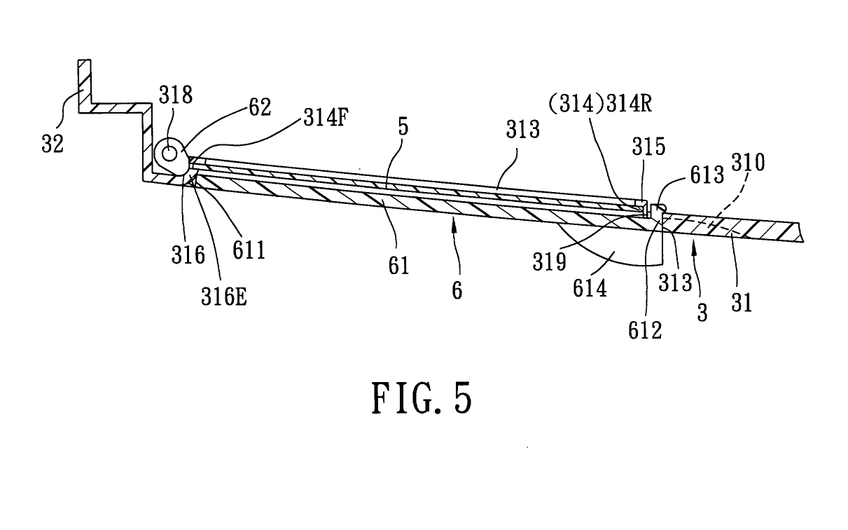

The window-confining wall 314 has a front wall portion 314F (see FIG. 5) proximate to the front part 32 and a rear wall portion 314R opposite to the front wall portion 314F. The front part 32 of the mask body 3 has a leaf portion 316 that is disposed adjacent to the front wall portion 314F of the window-confining wall 314, and that has a bent end 316E (see FIG. 5) projecting transversely into the side window 313 and cooperating with the abutting flange 315 to define a gap therebetween. The side viewing member 5,5′ has a front end formed with a protrusion 52 (see FIG. 2) extending into the gap so as to be sandwiched between the bent end 316E of the leaf portion 316 and the abutting flange 315 and so as to prevent untimely removal of the side viewing member 5, 5′ from the side window 313.

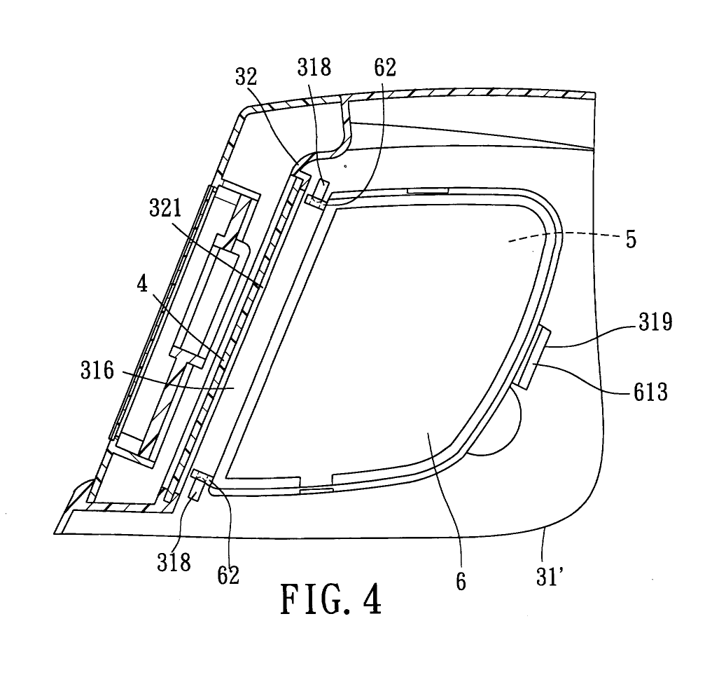

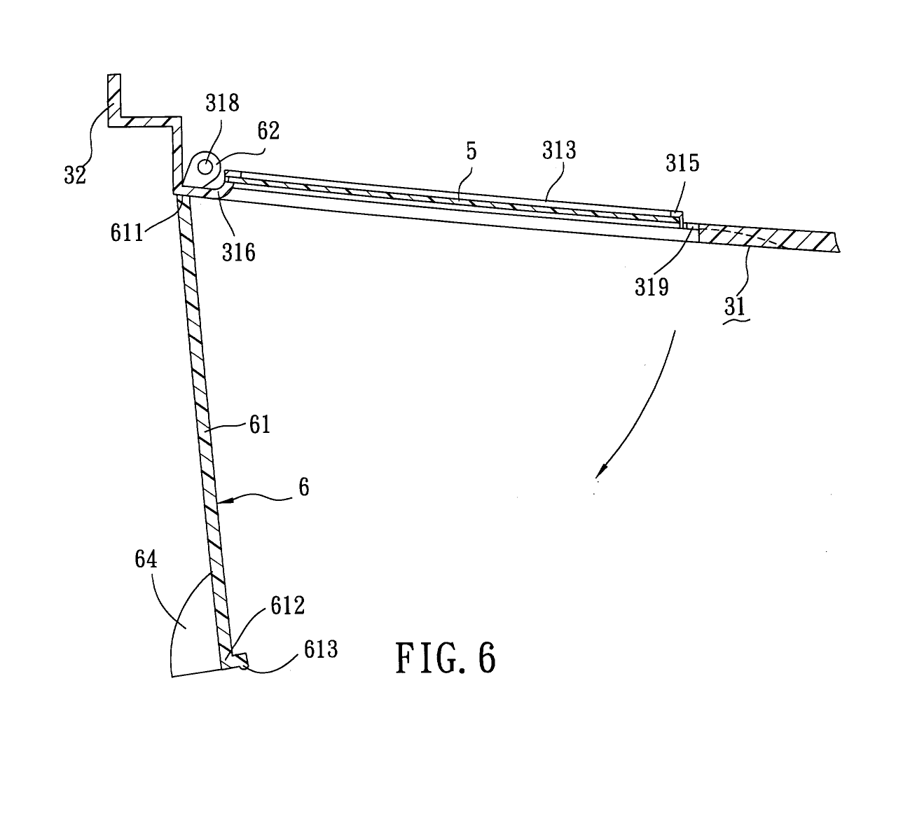

Each of the side covers 61 has a free end 612 and a pivot end 611 opposite to the free end 612. The pivot end 611 of each of the side covers 61 has spaced apart pivot ears 62 pivoted to the leaf portion 316 of the front part 32 through pivot pins 318 (see FIG. 4) in such a manner that the side cover 61 is movable between an open position, as best shown in FIG. 6, in which the free end 612 of the side cover 61 is moved away from the side window 313 so as to permit vision through the side window 313, and a closed position, as best shown in FIG. 5, in which the free end 612 of the side cover 61 is disposed in the side window 313 so as to cover the side viewing member 5.

Preferably, a groove-and-tongue assembly includes a tongue-engaging groove 319 formed in the rear wall portion 314R of the window-confining wall 314 of the side window 313, and an engaging tongue 613 protruding laterally and outwardly from the side cover 61 to engage the groove 319 for positioning the side cover 61 at the closed position.

Each of the left and right side parts 31,31′ of the mask body 3 is further formed with a finger-receiving recess 310 adjacent to the side window 313. Each of the side covers 61 has an outer surface formed with a protrusion 614 adjacent to the free end 612 thereof and confronting the finger-receiving recess 310 so as to facilitate opening of the side cover 61 by the user's finger.

With the provision of two side windows 313 in the mask body 3, the user of the protective mask 2 of the present invention can have a wide viewing range so that the aforesaid disadvantage of the prior art can be overcome accordingly.

With this invention thus explained, it is apparent that numerous modifications and variations can be made without departing from the scope and spirit of this invention. It is therefore intended that the invention be limited only as indicated in the appended claims.

I claim:

1. A protective mask comprising:
a mask body including left and right side parts and a front part disposed frontwardly and transversely of and interconnecting said left and right side parts, said front part being formed with a front window, at least one of said left and right side parts being formed with a side window;
a front viewing member attached to said front part and spanning said front window to cover said front window; and
a side viewing member attached to a respective one of said left and right side parts and spanning said side window to cover said side window.

2. The protective mask as defined in claim 1, wherein said side window is defined by a window-confining wall, said mask body being formed with an abutting flange projecting from said window-confining wall in a transverse direction relative to said window-confining wall into said side window, said side viewing member abutting against said abutting flange.

3. The protective mask as defined in claim 2, wherein said window-confining wall has a front wall portion proximate to said front part, said front part having a leaf portion that is disposed adjacent to said front wall portion of said window-confining wall, and that has a bent end projecting transversely into said side window and cooperating with said abutting flange to define a gap therebetween, said side viewing member having a front end formed with a protrusion extending into said gap so as to be sandwiched between said bent end of said leaf portion and said abutting flange.

4. The protective mask as defined in claim 3, further comprising a side cover with a free end and a pivot end opposite to said free end and pivoted to said leaf portion of said front part in such a manner that said side cover is movable between an open position, in which said free end of said side cover is moved away from said side window so as to permit vision through said side window, and a closed position, in which said free end of said side cover is disposed in said side window so as to cover said side viewing member.

5. The protective mask as defined in claim 4, wherein said window-confining wall of said side window further has a rear wall portion opposite to said front wall portion, said protective mask further comprising a groove-and-tongue assembly formed on said side cover and said rear wall portion of said window-confining wall of said side window for positioning said side cover at said closed position.
